(19) United States
(12) Patent Application Publication
(10) Pub. No.: US 2017/0180959 A1
(43) Pub. Date: Jun. 22, 2017

(54) ANIMAL MOBILE PHONE SERVICE SYSTEM AND METHOD

(71) Applicant: Myunghwan KIM, Soowon (KR)

(72) Inventor: Myunghwan KIM, Soowon (KR)

(21) Appl. No.: 15/316,506

(22) PCT Filed: Aug. 8, 2014
(86) PCT No.: PCT/KR2014/007380

§ 371 Date: Dec. 5, 2016

(30) Foreign Application Priority Data
Jun. 5, 2014 (KR) 10-2014-0068404

(51) Int. Cl. H04W 4/16; H04W 4/02; A01K 15/02; A01K 27/00; A01K 11/00; G06F 19/00; A01K 29/00

(52) U.S. Cl. CPC H04W 4/16; G06F 19/322; H04W 4/02; A01K 29/005; A01K 27/009; A01K 27/001; A01K 11/008; A01K 15/023

(57) ABSTRACT

The present invention relates to an animal mobile phone service system and method. The system comprises: a mobile terminal attached to a pet; an AMS management server for interworking with the mobile terminal attached to the pet, a mobile communication terminal of an owner or a guardian of the corresponding pet, and an AMS management server of a mobile communication network, the AMS management server providing an animal mobile phone service (AMS) including a method of training eating and bowel habits for a pet, music playing for a pet, identifying a location of a pet, a guardian notification function when a pet strays out of a predetermined zone after setting an active zone of the pet, and a vaccination and medical history management for a pet; a GIS map server for interworking with the AMS management server, displaying current location information of the mobile terminal attached to the pet, collecting a frequently moving path, and when the pet is missing or strays out of the predetermined zone, remotely tracing location information of the mobile terminal attached to the pet by providing an LBS service on a GIS map; and a text message server for, when the pet is missing or strays out of the predetermined zone, transmitting a warning message from the AMS management server to a mobile phone number of the mobile communication terminal of the owner or the guardian and generating a warning sound. Wherein the system provides, from the mobile communication terminal of the owner or the guardian of the animal to the mobile terminal of a pet, a remote voice communication service for a remote communication, a conversation with an animal, and a pet training to enable a pet or a military dog to perform a certain purpose, using human to animal (H2A) function.

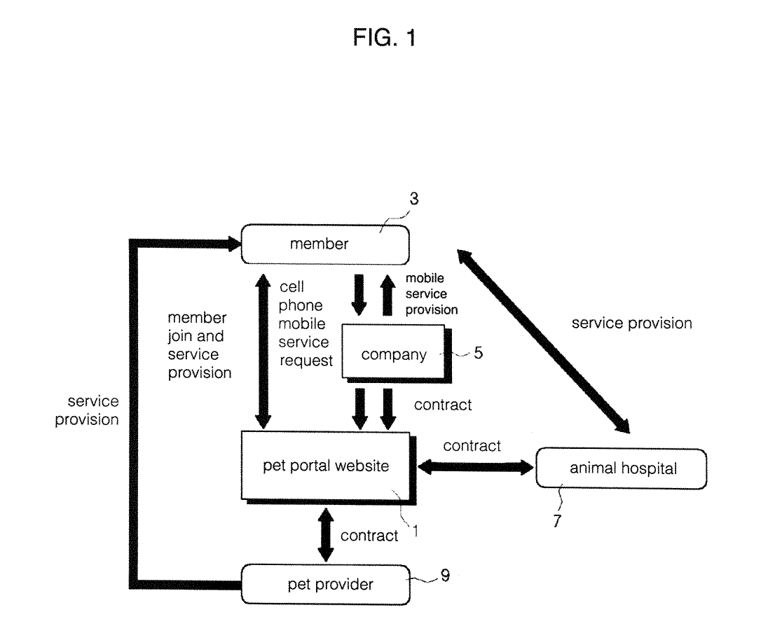

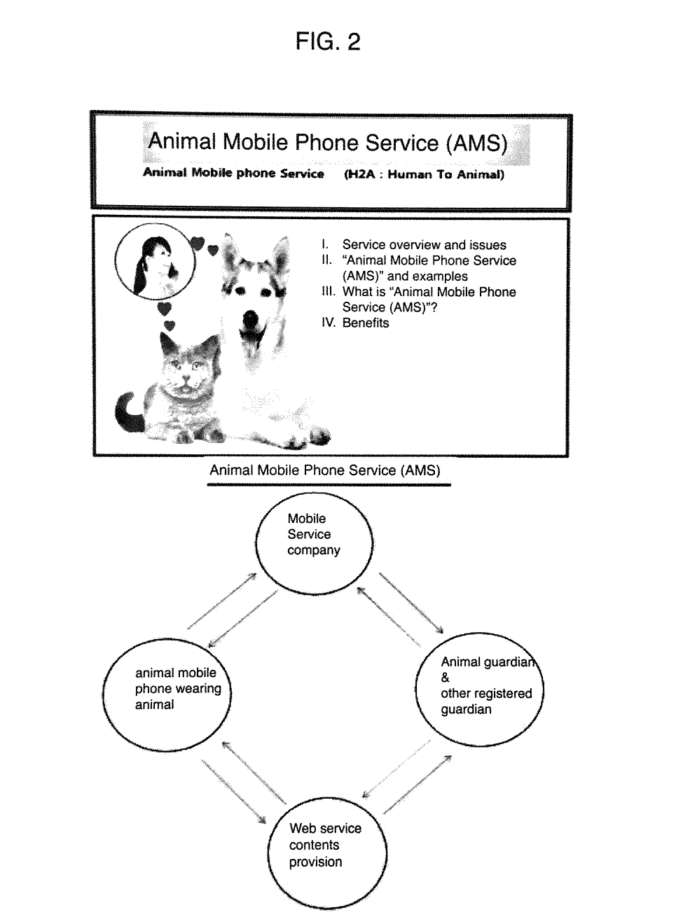

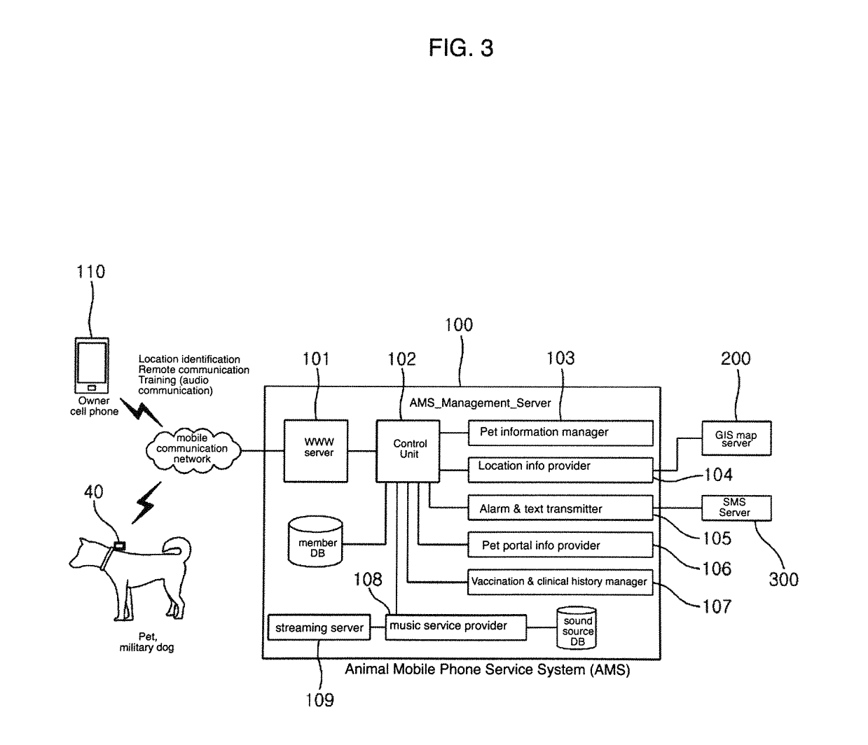

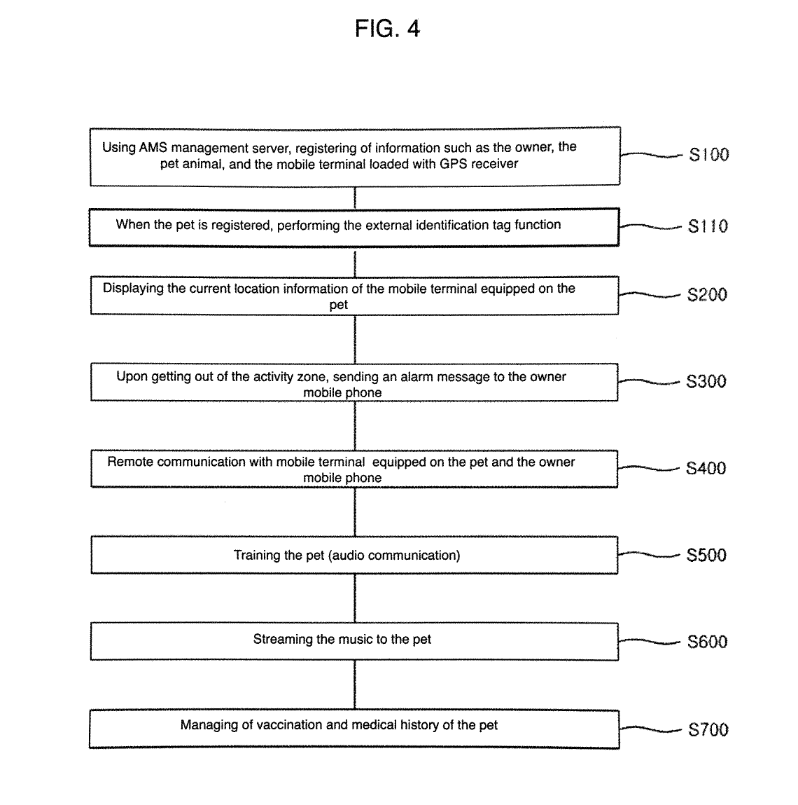

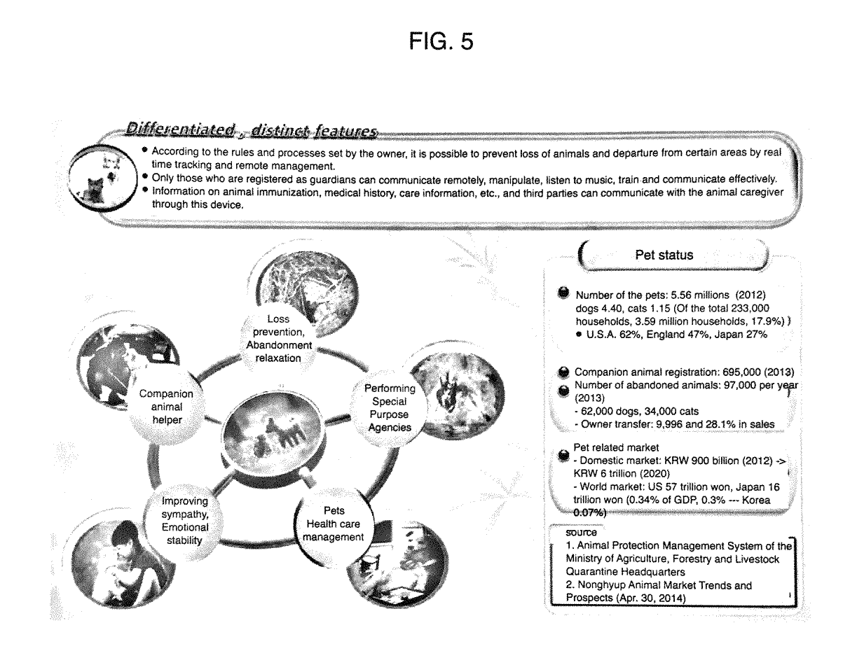

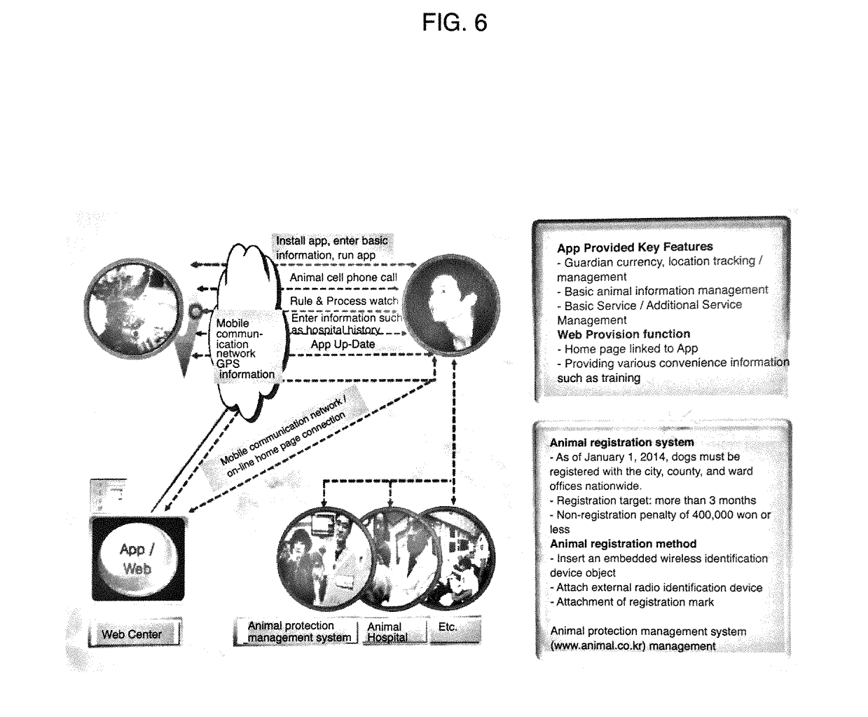

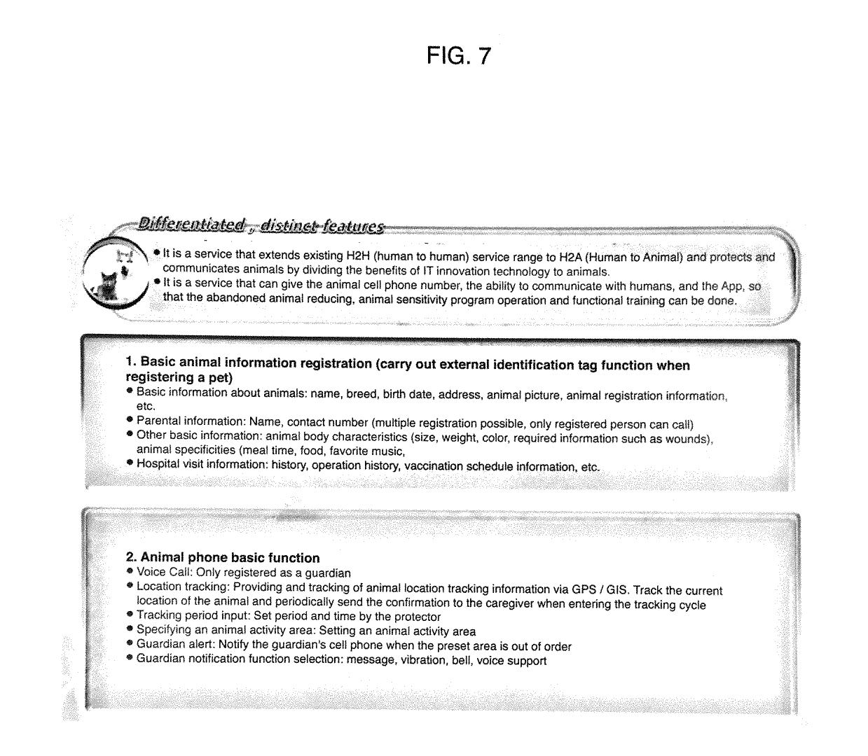

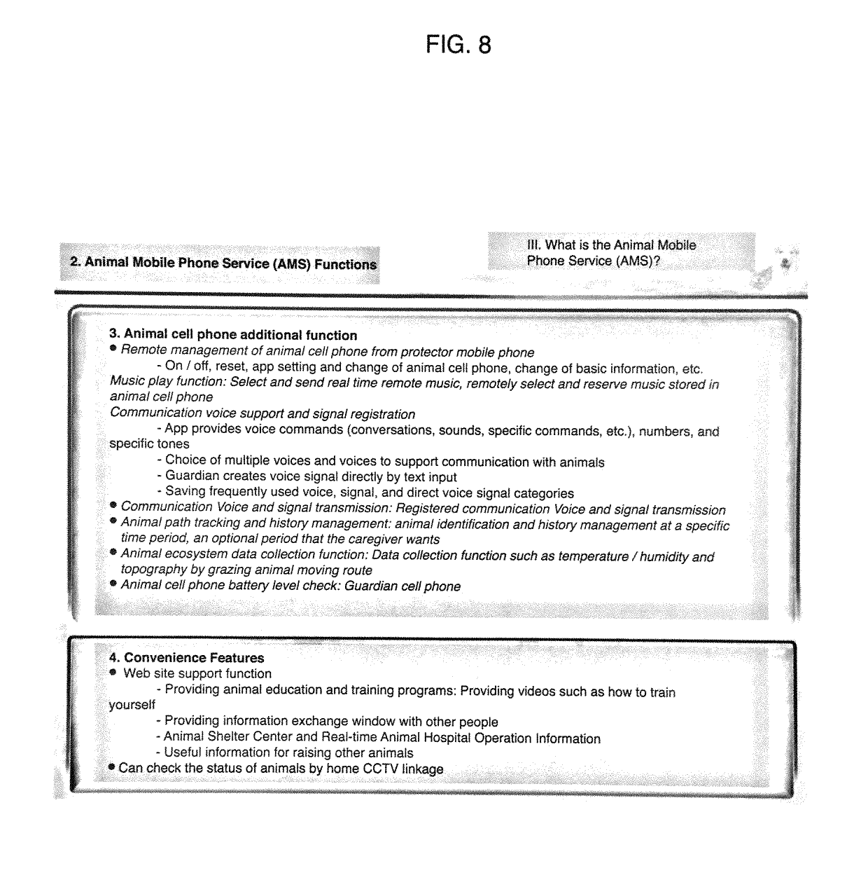

### TECHNOLOGY FIELD

This present invention relates to an animal mobile phone service system and method, where, in more detail, a mobile terminal (smartphone, wearable device, mobile device) with GPS receiver is equipped on the pet collar or a body part, and the service is linked to the mobile communication terminal of the corresponding animal guardian and the AMS management server of the mobile communication network, and in order to enable a pet or a military dog to perform a certain purpose, using human to animal (H2A) function, the service is controlled by a remote call or a certain signal to the mobile terminal of the pet from the mobile communication terminal of the owner or the guardian of the animal, and the service provides animal mobile phone service (AMS) such as conversation with the animal, playing music, training, pet location confirmation, guardian notification function in case of straying away from a predetermined zone, and management of vaccination and medical history of the pet, also providing LBS service on GIS map in case the animal is lost or strays out of a predetermined zone for remotely tracking the location information and easily locating the animal.

### BACKGROUND TECHNOLOGY

As a precedent technology related to this, the ‘pet management system using a mobile phone’ published with patent publication number 10-2003-0026648 is initiated, and as illustrated in Drawing 1, the pet management system using a mobile phone establishes a website (1) on the Internet, contracts with a mobile communication provider (5) in order to provide mobile service to a subscriber of the pet portal website mentioned above (1) as a member (3) joins the above website (1), provides mobile service for pets to the member through the telecommunication company (5) at the pet portal website (1) when the member (1) subscribes to the mobile phone service at the telecommunication company, and the member (3) provides pet care information to the pet portal website and then the above information is stored at the database of the pet portal website (1) to manage the pet.

The members are secured through the Internet at the operator server, and the database regarding the pet input by the subscriber is established and the information such as anniversaries, events, vaccinations, and meal time alarms are provided to the mobile phone of the subscriber through the telecommunication company without the need for the subscriber to go on the Internet, so as to provide pet management system using mobile phone for management of pets anywhere anytime.

However, with the existing system, it is impossible to have a conversation between the owner and the animal, to command or train remotely through voice or certain signals, and to perform an external identification tag function of animal protection registration system through basic information and other basic information of animal mobile phone in possession.

Also, the existing pet management system using mobile phone for managing pets does not provide LBS service function which tracks the location of pets remotely on GIS map in case the pet is lost or strays out of a predetermined zone, so it was difficult to find the pet, and it was impossible to notify the animal meal time through direct voice call or certain signals to the animal through the meal time notification function which was notified to the guardian only when providing food. Besides, the music playback function was not provided. Also, mobile phone voice support function for the handicapped and the training responding to certain signals were not included.

### DETAILED DESCRIPTION OF THE INVENTION

### Technological Task

The goal of this invention to resolve the above issues equips a mobile terminal (smartphone, wearable device, mobile device) loaded with GPS receiver on the pet collar or body part, links to the mobile communication terminal of the owner or corresponding animal guardian and the AMS management server of the mobile communication network, registers animal's basic information, guardian information, other basic information, hospital visit information and such within the mobile phone through H2A (Human to Animal) function, controls through remote calls or certain signals from the mobile communication terminal of the owner or the animal guardian to the mobile terminal of the pet for the pet or the military animal to perform a certain purpose, provides animal mobile phone service (AMS) such as conversation with the animal, playing music, training, pet location confirmation, animal activity zone designation, guardian notification function in case of straying away from a predetermined zone (message, vibration, bell, designated voice support), and vaccination and medical history management of pets, provides LBS (location based service) on GIS map in case an animal is lost or strays away from a predetermined zone by setting the animal activity zone so as to track the location information remotely for easy search, thus providing animal mobile phone service system.

Another goal of this invention is to equip a mobile terminal loaded with GPS receiver on the pet collar or body part so as to provide animal mobile phone service method.

### Means of Task Resolution

In order to achieve the goal of this invention, the animal mobile phone service system includes a mobile terminal equipped on the pet; the AMS management server which links to the above mobile terminal on the pet and the mobile communication terminal of the owner or the corresponding animal guardian and the AMS management server of the mobile communication network, and provides AMS such as method to train eating and toilet use of the pet, music playback for pets, pet location confirmation, guardian notification function in case of straying away from a predetermined zone, and vaccination and medical history management of pets; GIS map server which links to the above AMS management server, displays the current location information of the above mobile terminal equipped on the pet, collects the frequently travelled paths, provides LBS on GIS map which remotely tracks the location information of the above mobile terminal equipped on the pet in case an animal is lost or strays away from a predetermined zone; the text message server which sends an alarm message to the mobile phone number of the above mobile communication terminal of the owner or the guardian from the above AMS management server and generates an alarm sound in the form of message, designated voice, bell, or vibration for a certain period of time in case the above pet is lost or strays away from a predetermined zone by setting the animal activity zone, and provides the remote voice call service for training the pet, conversation with animals, remote call or certain signal to the above mobile terminal equipped on the pet from the mobile communication terminal of the owner or the guardian for the pet, police dog, or military dog to perform a certain purpose with the H2A (Human to Animal) function.

The above mobile terminal loaded with GPS receiver equipped on the pet shall use either a smartphone loaded with GPS receiver, a wearable device loaded with mobile communication voice call function and GPS receiver, or a separate mobile device loaded with mobile communication voice call function and GPS receiver, transfers to the above AMS management server the location tracking period of the above mobile terminal equipped on the pet arbitrarily configured by the guardian, and sprays a fluorescent substance for easy visibility at night. Also, the battery state of the animal mobile phone can be checked through the mobile phone of the guardian

The above mobile communication terminal of the owner or the guardian shall provide the remote animal mobile phone basic information registration function, guardian information registration function, other basic information registration function, hospital visit information, animal mobile phone remote management function, remote animal mobile phone on/off function, remote animal mobile phone reset function, remote animal mobile phone APP configuration and change function from the terminal itself to the above AMS management server, and is characteristic in its function for real-time remote music playback function to the mobile terminal equipped on the pet through the above AMS management server from the mobile communication terminal of the guardian whenever music or sound preferred by the animal is desired and the function to select music stored on the animal mobile phone and to configure time reservation from the mobile communication terminal of the owner or the guardian, the function for voice support for communication with the pet and sound registration [voice command (conversation, sound, certain commands), number, certain signal sounds are basically provided in the app], the function to register and store the voice signal directly generated by the guardian in the above AMS management server separately per animal category, the function to select multiple voices and tones supporting the communication with the pet, the function to transfer certain signals or communication commands stored per animal category, and the function to check the remaining battery by acquiring battery information of the animal mobile phone from the guardian mobile phone.

The above AMS management server further includes the vaccination and medical history register which manages vaccination and medical history management information of pets by renewing and registering the pet hospital visit information at the above AMS management server in case of pet treatment.

In order to achieve the other goal of this invention, the animal mobile phone service method includes (a) the phase where AMS management server receives the registration of the information of the owner or the guardian of the corresponding animal, the basic information of the pet and other basic information, the information of the music preferred by the pet and the guardian information, hospital visit information, and the information of the mobile terminal loaded with GPS receiver equipped on the pet through wired/wireless internet; (b) the phase where the above AMS management server provides pet portal service contents and food and training method information for the pet; (c) the phase where the current location information of the above mobile terminal equipped on the pet is displayed on GIS map on the mobile communication terminal of the above owner or the guardian of the corresponding animal; (d) the phase where the remote call data is transferred from the mobile communication terminal of the above owner or the above corresponding animal guardian to the mobile terminal equipped on the pet through the mobile communication network; and (e) the phase where the voice call data is transmitted from the mobile communication terminal of the above owner or the above corresponding animal guardian to the mobile terminal equipped on the pet to train the pet.

The above phase (c) further includes the phase where in case the location information of the mobile terminal equipped on the pet strays away from a predetermined zone, an alarm message is sent to the mobile phone number of the mobile communication terminal (smartphone) of the owner or the corresponding animal guardian from the above AMS management server and an alarm sound is generated in the form of a message, designated voice, bell or vibration for a certain period of time.

The above method further includes the phase where in case the music information pre-registered by the owner or the guardian of the animal is selected, the pre-stored music data is encoded from the above management server and streamed to the mobile terminal equipped on the pet for playback of the music preferred by the pet.

The above method further includes the phase where in case of pet treatment, the pet vaccination and medical history management information is managed by renewing and registering the hospital visit information of the pet to the above AMS management server.

### Effect of Invention

The animal mobile phone service system and method according to this invention has the effect of equipping a mobile terminal (smartphone, wearable device, mobile device) loaded with GPS receiver to the pet collar or body part, links to the AMS management server of the mobile communication network and the mobile communication terminal of the owner or the guardian of the corresponding animal, controlling through remote calls or certain signals from the mobile communication terminal of the owner or the animal guardian to the mobile terminal of the pet for the pet or the military animal to perform a certain purpose, providing animal mobile phone service (AMS) such as conversation with the animal, playing music, training, pet location confirmation, and providing LBS on GIS map in case an animal is lost or strays away from a predetermined zone to track the location information remotely for an easy search.

The animal mobile phone service creates new hiring opportunities in manufacturing, service, and management sectors through animal mobile phones, and can quickly and efficiently respond to an animal loss and significantly reduces the risk of animal loss through real-time call and location confirmation in case the animal is home alone or the guardian is out for an extended period of time.

The consistent communication with the animal provides sentimental stability to the owner or other registered animal guardian s and intimacy with human to the animal regardless of time or place, and even if the animal is out of sight, the command can be remotely performed so as to perform special-purpose tasks of military dogs and such, allowing human to avoid risk as much as possible.

Illness protection management is available due to consistent pet vaccination and illness history management, and the animal can be nurtured into a friendly animal in a short period of time using education or training program on the animal.

Also, the animal mobile phone service can play the music and sound preferred by the animal to promote sentimental stability, and can be installed at livestock such as free-range livestock to collect movement path and surrounding environment data, which then can be utilized as animal nurturing resources.

The indirect effect includes the active prevention of deserting the animal companion through proximate lifestyle with human through the animal mobile phone, the expansion to the field of mobile phone miniaturization and animal-related wearable device application field, prevent animal abuse as the first step to the new paradigm of H2A (Human to Animal) and promotes awareness for respecting life, the contribution to the vitalization of policies such as animal registration system related to animal protection and welfare enhancement, the expansion to animal ecosystem investigation, and the enhancement of awareness for animal protection and the improvement of culture for respecting animals.

### BRIEF DESCRIPTIONS OF DRAWINGS

Drawing 1 is the block diagram of the existing pet management system using mobile phones.

Drawing 2 is the concept diagram of the animal mobile phone service (AMS).

Drawing 3 is the block diagram of the animal mobile phone service system according to this invention.

Drawing 4 is the flowchart showing the animal mobile phone service method according to the execution example of this invention.

Drawing 5 is the drawing showing the animal mobile phone service overview.

Drawing 6 is the major flowchart of the animal mobile phone service overview.

Drawing 7 is the drawing showing the usage examples (1,2) of the animal mobile phone service overview.

Drawing 8 is the drawing showing the usage examples (3,4) of the animal mobile phone service overview.

### Optimal Form for the Execution of the Invention

The following is a detailed description of the desirable execution example of this invention with reference to the attached drawing.

Drawing 2 is the concept diagram of the animal mobile phone service (AMS).

The animal mobile phone service system equips

equips a mobile terminal (smartphone, wearable device, mobile device) loaded with GPS receiver on the pet collar or body part, links to the mobile communication terminal of the owner or corresponding animal guardian and the AMS management server of the mobile communication network, controls through remote calls or certain signals from the mobile communication terminal of the owner or the animal guardian to the mobile terminal of the pet for the pet or the military animal to perform a certain purpose through H2A (Human to Animal) function, provides animal mobile phone service (AMS) such as conversation with the animal, playing music, training, pet location confirmation, and vaccination and medical history management of pets, provides guardian notification function in case of straying away from a predetermined zone (message, vibration, bell, designated voice support), and also provides LBS (location based service) on GIS map in case an animal is lost or strays away from a predetermined zone so as to track the location information of the mobile terminal equipped on the pet collar or body part remotely for easy search of the pet.

The animal mobile phone service (AMS) expands the existing H2H (Human to Human) service scope to H2A (Human to Animal) so as to protect and communicate with the animal in association with AMS management server of the mobile communication company through the mobile communication terminal (smartphone) of the owner or other registered animal guardian

In this business model, a mobile phone number is attributed to the animal, and the human communication function and animal mobile phone service app is equipped for animal desertion prevention, animal sensibility program action, and functional training.

Pet Basic Information Registration (External Identification Tag Function when Companion Animals are Registered)
Pet basic information
Name, breed, birthdate, address information, animal photograph
Guardian information
Guardian name, contact number (multiple registrations possible)
Animal mobile phone guardian registration function where only the designated guardian can make a call
Other additional information
Animal physical characteristics (size, weight, color, scar, etc.)
Animal meal time and food to avoid
Animal special matters (meal time, food to avoid, animal preference on music and sound, etc.)

### Hospital Visit Information

Medical history matters and operation history
Vaccination history and schedule information, etc.

### Animal Mobile Phone Basic Function

Remote voice call function
Function to allow remote call with animal mobile phone only for registered guardian s
Function to connect call to the registered guardian on the animal phone
Location tracking function
Regularly send the animal location tracking and confirmation details to the guardian
Animal location tracking map information through GPS/GIS
Location tracking period input (determined at the discretion of the guardian)
Function of notification to the mobile phone of the owner or the guardian in case the animal strays away from a predetermined activity zone (specific zone)
Guardian notification selection function
Message, vibration, bell, designated voice support, etc.

### Animal Mobile Phone Additional Function

Animal route tracking and history management
Check the animal location by the period desired by the guardian and manage with history
Animal mobile phone remote management function from the mobile phone of the owner or the guardian
Remote animal mobile phone on/off function
Remote animal mobile phone reset function
Remote animal mobile phone APP setting and change
Remote animal mobile phone basic information registration function
Function to play music or sound preferred by the animal
Function for the guardian to transmit real-time music to the animal mobile phone
Function for the guardian to select music stored on the animal mobile phone and configure time reservation
Communication voice support and signal registration function
Voice command, number, and certain signals are basically provided in the app [symbol]
Voice command: conversation, sound, certain commands, etc.
The guardian creates voice signals directly and registers separately
Function to store the frequently used app-provided voices or signals and self-generated voice signals per animal category
Communication voice and signal transfer
Function to select multiple voices and tones supporting communication with animal
Function to transfer communication commands or certain signals stored per animal category
Website support function
Provide animal education and training program: provide videos for self-training methods
Provide information exchange window with others
Animal shelter and real-time vet clinic operation information
Provide other useful information for caring for an animal
Animal ecosystem investigation data collection function
Function to collect temperature, humidity, geography and such data per free-range animal travel path
Animal mobile phone remaining battery capacity check
Can check the remaining battery capacity for animal mobile phone from the mobile phone of the guardian
Real-time update of animal shelter and real-time vet clinic operation information
Home CCTV association function to identify the state of the animal

Drawing 3 is the animal mobile phone service system block diagram according to this invention.

The animal mobile phone service system has the mobile communication terminal (smartphone) (11) of the owner or the animal guardian linked to the AMS management server of the mobile communication network and mobile terminal (40) loaded with GPS receiver equipped on the pet, equips a mobile phone (smartphone) loaded with GPS receiver on the pet with the H2A (Human to Animal) function, controls through remote call or certain signals to perform certain purposes such as military dogs, provides animal mobile phone service (AMS) such as conversation with the animal, playing music, training, pet location confirmation, animal activity zone designation, guardian notification function in case of straying away from a predetermined zone (message, vibration, bell, designated voice support), and vaccination and medical history management of pets, and provides LBS (location based service) on GIS map in case an animal is lost or strays away from a predetermined zone so as to track the location information remotely for easy search.

The animal mobile phone service system includes the mobile terminal equipped on the pet (40); AMS management server (100) which is linked to the above mobile terminal (40) loaded with GPS receiver on the pet and the mobile communication terminal (11) of the owner or the guardian of the pet, and provides animal mobile phone service (AMS) such as method to train eating and toilet use of the pet, music playback for pets, pet location confirmation, guardian notification function in case of straying away from a predetermined zone, and vaccination and medical history management of pets; GIS map server (200) which links to the above AMS management server (100) and displays the current location information of the above mobile terminal equipped on the pet, collects the frequently travelled paths, and provides LBS on GIS map which remotely tracks the location information of the above mobile terminal (40) equipped on the pet in case an animal is lost or strays away from a predetermined zone; the text message server (300) which sends an alarm message to the mobile phone number of the above mobile communication terminal of the owner or the guardian from the above AMS management server and generates an alarm sound in the form of message, designated voice, bell, or vibration for a certain period of time in case the above pet is lost or strays away from a predetermined zone by setting the animal activity zone, and provides the remote voice call service for training the pet, conversation with animals, remote call or certain signal to the above mobile terminal equipped on the pet from the mobile communication terminal of the owner or the guardian for the pet, police dog, or military dog to perform a certain purpose with the H2A (Human to Animal) function.

The above mobile terminal (40) equipped on the animal shall use either a smartphone loaded with GPS receiver, a wearable device loaded with mobile communication voice call function and GPS receiver, or a separate mobile device loaded with mobile communication voice call function and GPS receiver.

The above mobile terminal (40) equipped on the animal shall transfer to the above AMS management server the location tracking period of the above mobile terminal equipped on the pet arbitrarily configured by the guardian, and sprays a fluorescent substance for easy visibility at night.

The mobile communication terminal (110) of the owner or the guardian shall provide the remote animal mobile phone basic information registration function, animal mobile phone remote management function, remote animal mobile phone on/off function, remote animal mobile phone reset function, remote animal mobile phone APP configuration and change function from the terminal itself (110) to the AMS management server (100), and shall provide the function for real-time remote music playback function to the mobile terminal (40) equipped on the pet through the above AMS management server whenever music or sound preferred by the animal is desired and the function to select music stored on the animal mobile phone and to configure time reservation from the mobile communication terminal of the owner or the guardian, the function for voice support for communication with the pet and sound registration [voice command (conversation, sound, certain commands), number, certain signal sounds are basically provided in the app], the function to register and store the voice signal directly generated by the guardian in the AMS management server (100) separately per animal category, the function to select multiple voices and tones supporting the communication with the pet, and the function to transfer certain signals or communication commands stored per animal category.

AMS management server (100) includes WWW server (101) providing web service; control unit (102) which controls each function of animal mobile phone service (AMS); pet information register (103) which registers and manages the pet basic information, additional information, guardian information, other basic information, hospital visit information, and the animal registration number (mobile phone number) of the mobile terminal (40) equipped on the pet collar or body part; location information provision unit (104) which is linked to GIS map server (200) and provides the location information to the mobile terminal (40) equipped on the pet; alarm sound and message transmission unit (105) which, in case the location information of the above mobile terminal (40) equipped on the pet indicates that animal activity zone strays away from a predetermined zone, sends an alarm message from the above AMS management server (100) to the mobile phone number of the mobile communication terminal (smartphone) of the owner or the corresponding animal guardian and generates the alarm sound in the form of message, designated voice, bell, or vibration over a certain period time; pet portal information provision unit (106) which provides pet portal contents including pet training method according to voice and signal training method and certain purposes (house-guarding, hunting, searching for missing people, drug detection, explosive detection, etc.) by generating certain signals or voice commands in order to provide pet feed or food per the pet breed such as pet, police dog, or military dog during meal time at the pet training website; vaccination and medical history management unit (107) which renews/registers pet hospital visit information at the above AMS management server (100) in case of animal treatment and manages vaccination and medical history management information of the pet; music service provision unit (108) transmits the pre-stored music data preferred by the pet from the music DB of the above AMS management server (100) once the music information preferred by the pet pre-registered by the owner or the animal guardian with a meal time notification or event function; encoder and streaming server (109) which encodes the music data preferred by the pet provided by the above music server provision unit (108) and transmits to the mobile terminal (40) equipped on the pet by streaming; and the animal mobile phone service provision unit including member DB and music DB; and is linked to home CCTV association system that can identify the state of the animal in association to home CCTV.

Drawing 4 is the flowchart showing the animal mobile phone service method according to the execution example of this invention.

The animal mobile phone service method according to the execution example of this invention includes the phase S100) where the information of the owner or the corresponding animal guardian (name, resident registration number, address, mobile phone number, e-mail address, etc.), pet basic information and other basic information, guardian information and information of music preferred by the pet, the hospital visit information, and the information of mobile terminal loaded with GPS receiver equipped on the pet at the AMS management server (100); the phase (S110) which performs the external identification tag function when the pet is registered;

the phase where the above AMS management server (100) provides pet portal service contents and provides pet training method information according to voice and signal training method and certain purposes (house-guarding, hunting, searching for missing people, drug detection, explosive detection, etc.) by generating certain signals or voice commands in order to provide pet feed or food per the pet breed such as pet, police dog, or military dog during meal time at the pet training website; the phase (S200) where the current location information of the mobile terminal (40) equipped on the pet on the mobile communication terminal (smartphone) is displayed on GIS map; the phase (S300) where, in case the location information of the above mobile terminal (40) equipped on the pet indicates that animal activity zone strays away from a predetermined zone, the above AMS management server (100) sends an alarm message to the mobile phone number of the mobile communication terminal (smartphone) of the owner or the corresponding animal guardian and generates the alarm sound in the form of message, designated voice, bell, or vibration over a certain period time; the phase (S400) where the remote call data is transmitted to the mobile terminal (40) equipped on the pet through the mobile communication network from the mobile communication terminal (smartphone) of the owner or the corresponding animal guardian; the phase (S500); where the voice call data is transmitted from the mobile communication terminal (smartphone) of the owner or the corresponding animal guardian to the mobile terminal (40) equipped on the pet so as to train the pet; the phase (S600) where, once the music data preferred by the pet pre-registered by the owner or the animal guardian is selected, with a meal time notification or event function, the music data pre-stored on the database of AMS management server (100) is encoded and sent to the mobile terminal (40) equipped on the pet by streaming and the music preferred by the pet is played; the phase (S700) which manages vaccination and medical history management information of pet by renewing and registering the pet hospital visit information at the AMS management server (100) in case of the pet treatment.

Drawing 5 is the drawing which shows the animal mobile phone service overview.

Drawing 6 is the animal mobile phone service major flow chart.

It provides App-provided major functions and Web-provided major functions, and the animal is attached with an animal identification tag by inserting an embedded wireless identification device entity is inserted into the mobile terminal loaded with GPS receiver or by attaching an external wireless identification device, and it is applied by registering to the animal protection management system at the vet clinics or other related institutions.

### AMS Service Overview

[symbol] It equips a mobile phone (smartphone) loaded with GPS receiver onto a pet, a military dog or such, remotely manages by displaying their locations on GIS map in real-time by the rules and processes predetermined by the owner, prevents the animal from being lost or straying away from a predetermined zone, and makes it easy to identify the owner when deserted or lost by registering animal basic information. [symbol]
It provides the animal mobile phone service (AMS) where only the person registered as the guardian (owner) can make a remote call or control with certain signals or use the embedded APP function, for effective management including conversation with the animal, music playback, training, and the pet vaccination and medical history management, and for communication with the pet. [symbol]
A third party can use this device to call their guardian s.
Animal Protection and Communication Pending Issue (if the Pet is Safe and More Friendly with Me . . . )
Is my pet well at home alone?
There is no way of finding out if it is well at home.
Is the pet outside playing all good?
It is difficult to know where it is, and you need to go out yourself and find it when necessary.
Can I quickly find a lost pet?
There is almost no way to find it unless someone fortunately finds them.
Can I have more conversations with my pet?
There is no way to talk to the pets playing far away.
There is no way to talk to the pets when far apart and you miss them.
Does my pet know that I am always there and loving?
It is impossible to give the sense that the owner is always there or to have a conversation frequently.
I want to play good music for my pet.
There are very limited ways to play music real-time conveniently.
I would like to educate efficiently and communicate conveniently
The education method and means are lacking.
There is no environment for frequent training.
I would like to control with remote command without needing to search for the animal
There is no way to call the pet real-time and demand a certain activity.
For a certain training (special-purpose dog, etc.)
There is no way for human and animal to act together under mutual communication through remotely controlling guide dog, drug detection dog, military dog, police dog and such certain purpose and the subsequent animal reaction of the animal. For example, the animal trained to perform a certain purpose according a certain guideline by training experts include military dogs for explosive treatment of dangerous areas in military bases, police dogs trained for drug detection performing narcotic control inspection, and guide dog for the blind.
I would like to efficiently manage my pet's vaccination and medical history management.

Drawing 7 is the drawing which shows the animal mobile phone service (AMS) usage example (1,2).

Drawing 8 is the drawing which shows the animal mobile phone service (AMS) usage example (3,4).

### II. Animal Mobile Phone Service (AMS) Usage Example

### Usage Example 1

### (In Case an Office Worker Living Alone has a Pet Dog A)

Mr. Kim, an office worker, has a lot of work recently and is getting off work later and later.

He is constantly worried; is his pet dog, adopted a while ago, getting lonely? Is it doing fine by itself? Is it eating well? A few days ago, he purchased an animal mobile phone and equipped it to his beloved pet. As soon as he gets to work, he calls and greets A. A responds as if he is right there, and after the call, he can feel safe and concentrate on the work. During lunchtime, A, who is already trained by the training app, is notified of the meal time through a certain signal, and heads to the auto-feeder. After lunch, the location confirmation shows that A is well at home. He selects and plays music that A likes, and imagines A having fun with its toys.

Getting home after work, he feels like he has been with A the whole time, and feels closer to it these days.

### Usage Example 2

### (In Case of a Police Dog B Special-Trained by the Animal Mobile Phone)

B, who has an animal mobile phone, always ranks at the top above other police dogs. Recently, it participated in a search for a missing person, and was awarded a commendation for searching the largest area. Unlike other police dogs, it performs the return order through the animal mobile phone, and can immediately move to another sector through remote commands.

Today, it was sent in alone for a dangerous article search under the subway. B immediately noticed the dangerous article and sent the voice through the animal mobile phone, notifying the police. The police remotely ordered B to clear from the area right away, and sent in the explosives handling unit to remove the explosive safely and quickly.

### Usage Example 3

(In Case of Going Out with a Pet Dog C)

It is fun to go out with C, but it is also tense because it may get lost at any time. You need to go get the groceries with C and go to the country house. The animal mobile phone has been equipped on the pet collar or body part, and you go in to the grocery store. C cannot enter, and has been tied outside. While doing groceries, you call C to keep reminding that the owner is here, and C can quietly wait for the owner outside. At a large empty field in the country, with no one else, you let C off the leash and have fun. It suddenly goes out of sight, but you can regularly check where C is through the animal mobile phone, and in case the pet strays away from the predetermined zone, an alarm occurs at the smartphone of the owner or the animal guardian. Soon, you call C and command it to return to the original location. If lost, you can easily find the pet with the animal registration number embedded in the animal mobile phone, allowing you to rest easy.

### Usage Example 4

(In Case a Person with a Speech Disorder has Adopted a Pet Dog D)

Mr. Lee, who has a speech disorder, always wanted to adopt a pet, but kept postponing due to the difficulties in communication with animals.

As soon as he learned that the animal mobile phone can substitute, he finally decided to adopt and purchase the animal mobile phone as well. He chose one of the dozens of voice items and was able to communicate with the animal through the mobile phone. Also, he was able to train D to keep the meal time by generating certain signals, and taught D to follow simple commands. Through remote voice calls, there was no need to worry when going out. He saw the voice and signal training method at the animal training website, and taught D one by one, and he grew more and more attached to D, who followed along very well, and felt grateful for the animal mobile phone.

As described above, it was explained with reference to the desirable execution example of this invention, but this invention can be modified or altered and executed within the scope of the technological idea and area of this invention indicated on the following patent claim scope, by someone with conventional knowledge in the technological field to which this invention belongs.

| | |
|---|---|
| 100: AMS management server | 101: WWW server |
| 102: control unit | 103: pet information management unit |
| 104: location information | 105: alarm sound and message |
| provision unit | transmission unit |
| 106: pet portal information | 107: vaccination and medical |
| provision unit | history management unit |
| 108: music service provision | 109: streaming server |
| unit | |

110: mobile phone of the owner or the animal guardian
40: mobile terminal loaded with GPS receiver equipped on the pet

| | |
|---|---|
| 200: GIS map server | 300: SMS server |

### INDUSTRIAL USABILITY

The animal mobile phone service system and method according to this invention has the effect of equipping a mobile terminal (smartphone, wearable device, mobile device) loaded with GPS receiver to the pet collar or body part, links to the AMS management server of the mobile communication network and the mobile communication terminal of the owner or the guardian of the corresponding animal, controlling through remote calls or certain signals from the mobile communication terminal of the owner or the animal guardian to the mobile terminal of the pet for the pet or the military animal to perform a certain purpose, providing animal mobile phone service (AMS) such as conversation with the animal, playing music, training, pet location confirmation, and providing LBS on GIS map in case an animal is lost or strays away from a predetermined zone to track the location information remotely for an easy search.

The animal mobile phone service creates new hiring opportunities in manufacturing, service, and management sectors through animal mobile phones, and can quickly and efficiently respond to an animal loss and significantly reduces the risk of animal loss through real-time call and location confirmation in case the animal is home alone or the guardian is out for an extended period of time.

The consistent communication with the animal provides sentimental stability to the owner or other registered animal guardian s and intimacy with human to the animal regardless of time or place, and even if the animal is out of sight, the command can be remotely performed so as to perform special-purpose tasks of military dogs and such, allowing human to avoid risk as much as possible.

Illness protection management is available due to consistent pet vaccination and illness history management, and the animal can be nurtured into a friendly animal in a short period of time using education or training program on the animal.

Also, the animal mobile phone service can play the music and sound preferred by the animal to promote sentimental stability, and can be installed at livestock such as free-range livestock to collect movement path and surrounding environment data, which then can be utilized as animal nurturing resources.

The indirect effect includes the active prevention of deserting the animal companion through proximate lifestyle with human through the animal mobile phone, the expansion to the field of mobile phone miniaturization and animal-related wearable device application field, prevent animal abuse as the first step to the new paradigm of H2A (Human to Animal) and promotes awareness for respecting life, the contribution to the vitalization of policies such as animal registration system related to animal protection and welfare enhancement, the expansion to animal ecosystem investigation, and the enhancement of awareness for animal protection and the improvement of culture for respecting animals.

1. The animal mobile phone service system which includes mobile terminal equipped on the pet;
AMS management server which is linked to the above mobile terminal on the pet and the mobile communication terminal of the owner or the corresponding animal guardian through the mobile communication network, and provides animal mobile phone service (AMS) such as method to train eating and toilet use of the pet, music playback for pets, pet location confirmation, guardian notification function in case of straying away from a predetermined zone, and vaccination and medical history management of pets;
GIS map server which links to the above AMS management server, displays the current location information of the above mobile terminal equipped on the pet, collects the frequently travelled paths, provides LBS on GIS map which remotely tracks the location information of the above mobile terminal equipped on the pet in case an animal is lost or strays away from a predetermined zone;
text message server which sends an alarm message to the mobile phone number of the above mobile communication terminal of the owner or the guardian from the above AMS management server and generates an alarm sound in case the above pet is lost or strays away from a predetermined zone;
and is characteristic in providing the remote voice call service for training the pet, conversation with animals, and remote call to the above mobile terminal equipped on the pet from the mobile communication terminal of the owner or the guardian for the pet, police dog, or military dog to perform a certain purpose with the H2A (Human to Animal) function.

2. With respect to claim 1, the animal mobile phone service system which is characteristic in that, with respect to Clause 1, the above mobile terminal loaded with GPS receiver equipped on the pet shall use either a smartphone loaded with GPS receiver, a wearable device loaded with mobile communication voice call function and GPS receiver, or a separate mobile device loaded with mobile communication voice call function and GPS receiver, transfers to the above AMS management server the location tracking period of the above mobile terminal equipped on the pet arbitrarily configured by the guardian, and sprays a fluorescent substance for easy visibility at night.

3. With respect to claim 1, the animal mobile phone service system which is characteristic in that the above mobile communication terminal of the owner or the guardian shall provide the remote animal mobile phone basic information registration function, guardian information registration function, other basic information registration function, hospital visit information, animal mobile phone remote management function, remote animal mobile phone on/off function, remote animal mobile phone reset function, remote animal mobile phone APP configuration and change function from the terminal itself to the above AMS management server, and is characteristic in its function for real-time remote music playback function to the mobile terminal equipped on the pet through the above AMS management server from the mobile communication terminal of the guardian whenever music or sound preferred by the animal is desired and the function to select music stored on the animal mobile phone and to configure time reservation from the mobile communication terminal of the owner or the guardian, the function for voice support for communication with the pet and sound registration [voice command (conversation, sound, certain commands), number, certain signal sounds are basically provided in the app], the function to register and store the voice signal directly generated by the guardian in the above AMS management server separately per animal category, the function to select multiple voices and tones supporting the communication with the pet, the function to transfer certain signals or communication commands stored per animal category, and the function to check the remaining battery by acquiring battery information of the animal mobile phone from the guardian mobile phone.

4. With respect to claim 1, the animal mobile phone service system which is characteristic in that the above AMS management server includes WWW server providing web service;
control unit which controls each function of animal mobile phone service (AMS);
pet information register which registers and manages the pet basic information, additional information, guardian information, other basic information, hospital visit information, and the animal registration number (mobile phone number) of the mobile terminal equipped on the pet collar or body part;
location information provision unit which is linked to GIS map server and provides the location information to the mobile terminal equipped on the pet;
alarm sound and message transmission unit which, in case the location information of the above mobile terminal equipped on the pet indicates that animal activity zone strays away from a predetermined zone, sends an alarm message from the above AMS management server to the mobile phone number of the mobile communication terminal (smartphone) of the owner or the corresponding animal guardian and generates the alarm sound in the form of message, designated voice, bell, or vibration over a certain period time;
pet portal information provision unit which provides pet portal contents including pet training method according to voice and signal training method and certain purposes (house-guarding, hunting, searching for missing people, drug detection, explosive detection, etc.) by generating certain signals or voice commands in order to provide pet feed or food per the pet breed such as pet, police dog, or military dog during meal time at the pet training website;
vaccination and medical history management unit which renews/registers pet hospital visit information at the above AMS management server in case of animal treatment and manages vaccination and medical history management information of the pet;
music service provision unit transmits the pre-stored music data preferred by the pet from the music DB of the above AMS management server once the music information preferred by the pet pre-registered by the owner or the animal guardian with a meal time notification or event function;
encoder and streaming server which encodes the music data preferred by the pet provided by the above music server provision unit and transmits to the mobile terminal equipped on the pet by streaming;
and the animal mobile phone service provision unit including member DB and music DB;
and is linked to home CCTV association system that can identify the state of the animal in association to home CCTV.

5. The animal mobile phone service method which includes
(a) the phase where AMS management server receives the registration of the information of the owner or the guardian of the corresponding animal, the basic information of the pet and other basic information, the information of the music preferred by the pet and the guardian information, hospital visit information, and the information of the mobile terminal loaded with GPS receiver equipped on the pet through wired/wireless internet;
(b) the phase where the above AMS management server provides pet portal service contents and food and training method information for the pet;
(c) the phase where the current location information of the above mobile terminal equipped on the pet is displayed on GIS map on the mobile communication terminal of the above owner or the guardian of the corresponding animal;
(d) the phase where the remote call data is transferred from the mobile communication terminal of the above owner or the above corresponding animal guardian to the mobile terminal equipped on the pet through the mobile communication network; and
(e) the phase where the voice call data is transmitted from the mobile communication terminal of the above owner or the above corresponding animal guardian to the mobile terminal equipped on the pet to train the pet.

6. With respect to claim 5, the animal mobile phone service method where the above phase (c) further includes the phase where in case the location information of the mobile terminal equipped on the pet strays away from a predetermined zone, an alarm message is sent to the mobile phone number of the mobile communication terminal (smartphone) of the owner or the corresponding animal guardian from the above AMS management server and an alarm sound is generated in the form of a message, designated voice, bell or vibration for a certain period of time.

7. With respect to claim 5, the animal mobile phone service method which further includes (f) the phase where in case the music information pre-registered by the owner or the guardian of the animal is selected, the pre-stored music data is encoded from the above management server and streamed to the mobile terminal equipped on the pet for playback of the music preferred by the pet.

8. With respect to claim 5, the animal mobile phone service method which further includes (g) the phase where in case of pet treatment, the pet vaccination and medical history management information is managed by renewing and registering the hospital visit information of the pet to the above AMS management server.
